(19) United States
(12) Patent Application Publication
(10) Pub. No.: US 2009/0306636 A1
(43) Pub. Date: Dec. 10, 2009

(54) DERMAL TREATMENT DEVICE

(76) Inventors: HAI BEN-ISRAEL, Yavne (IL); YEHOSHUA STEINBERG, Neve Zuf (IL)

Correspondence Address: DANIEL J SWIRSKY, 55 REUVEN ST., BEIT SHEMESH, 99544, IL

(21) Appl. No.: 12/365,905

(22) Filed: Feb. 5, 2009

Related U.S. Application Data
(60) Provisional application No. 61/026,478, filed on Feb. 6, 2008.

(51) Int. Cl. A61B 18/18; H05K 7/20

(52) U.S. Cl. 606/9; 361/694

(57) ABSTRACT

An applicator unit for a dermal treatment system using intense pulsed light, the applicator comprising: a compartment with an opening to the patient's skin, an optical filter unit dividing the compartment into a closed chamber and an open chamber, where the open chamber having the opening; a light-source unit mounted inside the closed chamber and operative to radiate light through the optical filter unit and through the opening on the patient's skin; and an air-cooling unit operative to cool the closed chamber.

### CROSS-REFERENCE TO RELATED APPLICATIONS

This present application claims priority from U.S. provisional patent application No. 61/026478 filed Feb. 6, 2008, the contents of which are hereby incorporated by reference.

### FIELD AND BACKGROUND OF THE INVENTION

The present invention relates to devices and methods for dermal treatment and, more particularly, but not exclusively to treatment of the skin using intense pulsed light.

The following US Pre-Grant Publications are believed to represent the most relevant prior art: US2005055073 and US20070159592.

Many current devices and methods use light for treating human skin, particularly for applications such as hair removal. Such devices use intense pulsed light, preferably in specific wavelengths, to effect specific skin cells, such as hair roots. The higher concentration of melanin in the hair root, compared to the surrounding tissue, absorbs the light and concentrates the heat in the root of the hair, thus disturbing hair growth.

The intense heat produced by current devices requires cooling of the radiated tissue. There are therefore many methods for cooling the tissue, including applying liquids, gel, cooled surfaces, etc. All these methods are time consuming and unpleasant to the patient.

There is thus a widely recognized need for, and it would be highly advantageous to have, a light-based dermal treatment device devoid of the above limitations.

### SUMMARY OF THE INVENTION

According to one aspect of the present invention there is provided a dermal treatment system including an applicator head, the applicator head including:
a compartment comprising an opening;
an optical filter unit dividing the compartment into a closed chamber and an open chamber, the open chamber comprising the opening;
a light-source unit mounted inside the closed chamber and operative to radiate light through the optical filter unit and through the opening; and
an air-cooling unit operative to cool the closed chamber.

According to another aspect of the present invention there is provided an applicator unit for the dermal treatment system, the applicator unit comprising:
a compartment comprising an opening;
an optical filter unit dividing the compartment into a closed chamber and an open chamber, the open chamber comprising the opening;
a light-source unit mounted inside the closed chamber and operative to radiate light through the optical filter unit and through the opening; and
an air-cooling unit operative to cool the closed chamber.

According to another aspect of the present invention there is provided an applicator unit for the dermal treatment system wherein the air-cooling unit comprises at least one of:
an opening operative to connect to a pipe pushing air into the closed chamber;
an opening operative to connect to a pipe sucking air from the closed chamber;
an opening operative to connect the closed chamber to ambient air;
an air-pump operative to push air via the pipe; and
an air-pump operative to suck air via the pipe.

According to yet another aspect of the present invention there is provided an applicator unit for the dermal treatment system, wherein the air-cooling unit is a fan mounted inside the closed chamber.

According to still another aspect of the present invention there is provided an applicator unit for the dermal treatment system, wherein the opening comprising a surrounding edge, the edge operative to provide contact with skin surface of a patient.

Further according to another aspect of the present invention there is provided an applicator unit for the dermal treatment system wherein the edge is operative to induce a mark of the edge boundary on the skin surface of the patient upon touching the skin surface.

Yet further according to another aspect of the present invention there is provided an applicator unit for the dermal treatment system wherein the edge comprises a line of teeth operative to induce the mark.

Still further according to another aspect of the present invention there is provided an applicator unit for the dermal treatment system wherein the edge is operative to secure a gap between the optical filter and the skin surface.

Even further according to another aspect of the present invention there is provided an applicator unit for the dermal treatment system wherein the gap is adjustable.

Unless otherwise defined, all technical and scientific terms used herein have the same meaning as commonly understood by one of ordinary skill in the art to which this invention belongs. The materials, methods, and examples provided herein are illustrative only and not intended to be limiting. Except to the extend necessary or inherent in the processes themselves, no particular order to steps or stages of methods and processes described in this disclosure, including the figures, is intended or implied. In many cases the order of process steps may varied without changing the purpose or effect of the methods described.

Implementation of the method and system of the present invention involves performing or completing certain selected tasks or steps manually, automatically, or any combination thereof. Moreover, according to actual instrumentation and equipment of preferred embodiments of the method and system of the present invention, several selected steps could be implemented by hardware or by software on any operating system of any firmware or any combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a simplified illustration of a closed-chamber intense-pulsed-light dermal-treatment device; and

FIG. 1B is an enlarged simplified illustration of a cut-through a closed-chamber applicator unit of the intense-pulsed-light dermal-treatment device.

### DETAILED DESCRIPTION OF THE INVENTION

The present embodiments comprise a device for treating the skin with intense pulsed light, and particularly, a closed-chamber optical-head for such device.

The principles and operation of a closed-chamber intense-pulsed-light dermal-treatment device according to the present invention may be better understood with reference to the drawings and accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In this document, an element of a drawing that is not described within the scope of the drawing and is labeled with a numeral that has been described in a previous drawing has the same use and description as in the previous drawings. Similarly, an element that is identified in the text by a numeral that does not appear in the drawing described by the text, has the same use and description as in the previous drawings where it was described.

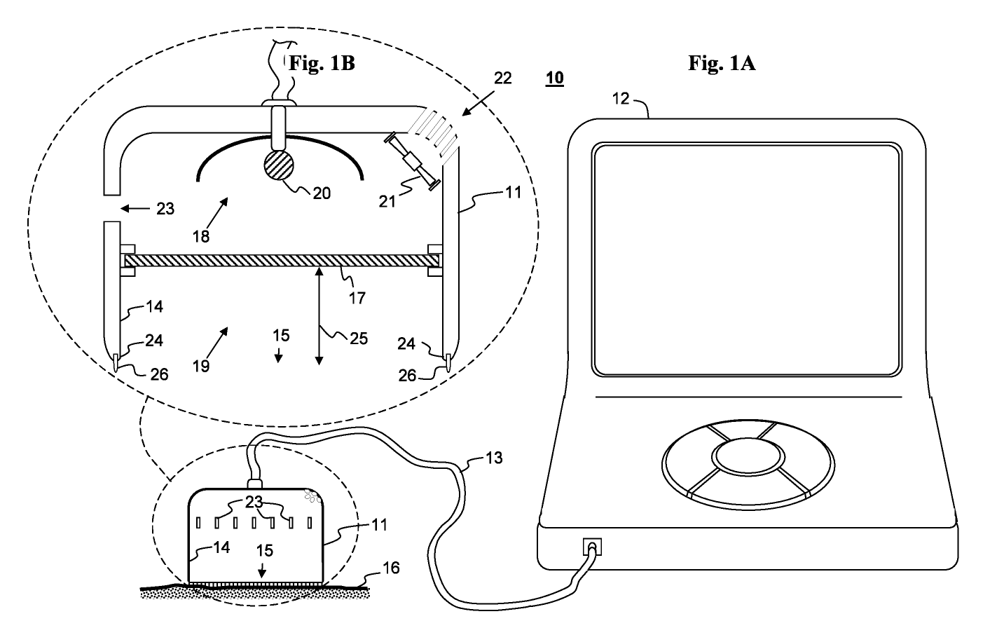

Reference is now made to FIG. 1A, which is a simplified illustration of a closed-chamber intense-pulsed-light dermal-treatment device 10, and to FIG. 1B, which is an enlarged simplified illustration of a cut through a closed-chamber applicator unit 11 of the intense-pulsed-light dermal-treatment device 10, according to a preferred embodiment of the present invention.

As seen in FIG. 1A, the closed-chamber intense-pulsed-light dermal-treatment device 10 preferably includes a control unit 12 connected via an electric wire 13 to the closed-chamber applicator unit 11.

As seen in FIG. 1B, The closed-chamber applicator unit 11 preferably includes:
a compartment 14, preferably in the form of a chamber or a cavity, with an opening 15 to be attached to patient's skin 16;
an optical filter unit 17 dividing the compartment into a closed part 18 and an open part 19, where the open part comprising the opening 15;
a light-source unit 20 mounted inside the closed part 18, preferably oriented to radiate light through the optical filter unit 17 and through the opening 15; and
an air-cooling unit 21 operative to cool the closed chamber 18.

As seen in FIGS. 1A and 1B the control unit 12 preferably provides electric power via electric wire 13 to operate the light source 20 to produce intense pulsed light. The pulsed light is filtered by the optical filter 17 to produce light radiation of limited frequency band, typically at the wavelength range of 550-1200 nanometers. It is appreciated that other wavelength ranges can be used according to the type of the required treatment.

The intense pulsed light produces high heat energy within the closed chamber 18, which heats the light source 20 and the optical filter 17 as well as the compartment 11. The air-cooling unit 21 preferably introduces ambient air into the closed chamber 18 to cool the closed chamber 18 as well as the light source 20, the optical filter 17 and the compartment 14. The air-cooling unit 21 is preferably implemented as a fan sucking air from the closed chamber 18 and blowing it outside through the ventilation slits 22. Air openings 23 can be provided to assist the flow of air through the closed chamber 18.

It is appreciated that, alternatively, the air-cooling unit 21 can operate in the reverse direction, sucking ambient air and blowing it inside the closed chamber 18.

As seen in FIGS. 1A and 1B, the air-cooling unit 21 is operative to cool the volume enclosed by the compartment 14 and the optical filter 17 (i.e. the closed chamber 18), and is not intended to cool the skin 16 and the volume enclosed between the compartment 14 and the optical filter 17 and the skin 16 (i.e. the open chamber 19.

It is appreciated that the air-cooling unit 21 may be configured in any of the following configurations:
Preferably, a ventilation opening in the closed chamber 18 and a fan mounted close to the ventilation opening, preferably inside the closed chamber 18.
An opening in the closed chamber 18 connecting to a pipe pushing air into the closed chamber 18; and an air-pump pushing air via the pipe; and
An opening in the closed chamber 18 connecting to a pipe sucking air into the closed chamber 18; and an air-pump sucking air via the pipe.

It is appreciated that the compartment 14 can have an edge 24 surrounding the opening 15 and that the edge is configured to provide contact with skin surface of a patient. It is appreciated that the edge can be configured to secure a gap 25 between the optical filter and the skin surface so that the optical filter does not touch the skin surface. It is appreciated that the gap is adjustable preferably according to the energy level of the radiated light.

Typically, a skin treatment comprises a series of flash-treatments. In each flash-treatment the person operating the intense-pulsed-light dermal-treatment device 10 places the applicator unit 11 on a part of the patient's skin. Preferably, the edge 24 contains a line of teeth 26 protruding from the edge in the direction of the skin. The pressure of the tips of the teeth 26 on the skin preferably leaves a mark on the skin designating where the perimeter of the edge 24 touched the skin of the patient. Thus, marking the area of the recent flash treatment. The perimeter mark enables the operator to place the applicator unit 11 for the next flash treatment on a part of the skin adjacent to the previously treated area.

It is expected that during the life of this patent many relevant types of dermal treatments will be developed as well as relevant devices and systems, and the scope of the terms herein, particularly of the terms “intense pulsed light”, “air-cooling” and “applicator”, is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An applicator unit for a dermal treatment system, the applicator unit comprising:
a compartment comprising an opening;
an optical filter unit dividing the compartment into a closed chamber and an open chamber, the open chamber comprising the opening;
a light-source unit mounted inside the closed chamber and operative to radiate light through the optical filter unit and through the opening; and
an air-cooling unit operative to cool the closed chamber.

2. An applicator unit according to claim 1 wherein the air-cooling unit comprises at least one of:
an opening operative to connect to a pipe pushing air into the closed chamber;
an opening operative to connect to a pipe sucking air from the closed chamber;
an opening operative to connect the closed chamber to ambient air;
an air-pump operative to push air via the pipe; and
an air-pump operative to suck air via the pipe.

3. An applicator unit according to claim 1 wherein the air-cooling unit is a fan mounted inside the closed chamber.

4. An applicator unit according to claim 1 wherein the opening comprising a surrounding edge, the edge operative to provide contact with skin surface of a patient.

5. An applicator unit according to claim 4 wherein the edge is operative to induce a mark of the edge boundary on the skin surface of the patient upon touching the skin surface.

6. An applicator unit according to claim 5 wherein the edge comprises a line of teeth operative to induce the mark.

7. An applicator unit according to claim 4 wherein the edge is operative to secure a gap between the optical filter and the skin surface.

8. An applicator unit according to claim 7 wherein the gap is adjustable.

9. A dermal treatment system comprising an applicator head, the applicator head comprising:
a compartment comprising an opening;
an optical filter unit dividing the compartment into a closed chamber and an open chamber, the open chamber comprising the opening;
a light-source unit mounted inside the closed chamber and operative to radiate light through the optical filter unit and through the opening; and
an air-cooling unit operative to cool the closed chamber.

10. A dermal treatment system according to claim 9 wherein the air-cooling unit comprises at least one of:
an opening operative to connect to a pipe pushing air into the closed chamber;
an opening operative to connect to a pipe sucking air from the closed chamber;
an opening operative to connect the closed chamber to ambient air;
an air-pump operative to push air via the pipe; and
an air-pump operative to suck air via the pipe;

11. A dermal treatment system according to claim 9 wherein the air-cooling unit is a fan mounted inside the closed chamber.

12. A dermal treatment system according to claim 9 wherein the opening comprising a surrounding edge, the edge operative to provide contact with skin surface of a patient.

13. A dermal treatment system according to claim 9 wherein the edge is operative to induce a mark of the edge boundary on the skin surface of the patient upon touching the skin surface.

14. A dermal treatment system according to claim 9 wherein the edge comprises a line of teeth operative to induce the mark.

15. A dermal treatment system according to claim 12 wherein the edge is operative to secure a gap between the optical filter and the skin surface.

16. A dermal treatment system according to claim 15 wherein the gap is adjustable.
